(19) United States
(12) Patent Application Publication
(10) Pub. No.: US 2018/0071365 A1
(43) Pub. Date: Mar. 15, 2018

(54) METHODS AND COMPOSITIONS FOR ENHANCING CARDIAC CONTRACTILITY FOR TREATMENT OF HEART FAILURE

(71) Applicant: NOVA SOUTHEASTERN UNIVERSITY, Fort Lauderdale, FL (US)

(72) Inventor: Anastasios LYMPEROPOULOS, Fort Lauderdale, FL (US)

(73) Assignee: NOVA SOUTHEASTERN UNIVERSITY, Fort Lauderdale, FL (US)

(21) Appl. No.: 15/554,721

(22) PCT Filed: Mar. 4, 2016
(86) PCT No.: PCT/US16/20812

§ 371 Date: Aug. 31, 2017

Related U.S. Application Data
(60) Provisional application No. 62/128,335, filed on Mar. 4, 2015.

(51) Int. Cl. A61K 38/17; C12N 15/861

(52) U.S. Cl. CPC A61K 38/1787; C12N 2710/10371; C12N 2710/10343; C12N 15/861

(57) ABSTRACT

The invention provides a therapeutically-effective method for treatment of heart failure (HF), chronic heart failure, and heart failure post myocardial infarction (MI). This method treats heart failure by enhancing cardiac contractility in a patient by activating β-arrestin 2 to enhance sarco(endo) plasmic reticulum Ca⁻² ATPase (SERCA-2a) small ubiquitin-like modifier-ylation (SUMOlation). Thus, β-arrestin 2 stimulates cardiac function in heart failure via SERCA-2a potentiation. Additionally, the invention provides a composition for increasing cardiac contractility including a β-1 adrenergic receptor (β-1 AR) ligand that induces β-arrestin 2 binding to a β-1 adrenergic receptor.

### FIELD OF THE INVENTION

The invention generally relates to methods and compositions for the treatment of heart failure (HF), particularly to methods and compositions for treating heart failure by enhancing cardiac contractility in a heart failure patient and/or any patient in need of enhanced cardiac contractility, and most particularly to methods and compositions for treating heart failure by enhancing cardiac contractility in a patient by activating β-arrestin 2 to enhance sarco(endo) plasmic reticulum Ca+² ATPase (SERCA-2a) small ubiquitin-like modifier-ylation (SUMOlation).

### BACKGROUND

Heart failure (HF) is the number one killer disease in the western world and new and innovative treatments are needed. Sarco(endo)plasmic reticulum Ca²⁺-ATPase (SERCA)-2a is a crucial, for contractile function, calcium-handling protein expressed in the mammalian myocardium and its downregulation is one of the molecular hallmarks of chronic HF (1). Its activation is part of the signaling mechanism by which the β₁-adrenergic receptors (ARs) increase cardiac contractility (2). Agonist-bound β₁ARs however, like most G protein-coupled receptors (GPCRs), undergo functional desensitization/internalization due to the actions of β-arrestin 1 or 2 (3). These two arrestins are universal GPCR adapter proteins, mediating G protein-independent signaling via multi-protein scaffolding (3), and, among the cellular processes they can regulate, is protein SUMO (small ubiquitin-like modifier)-ylation (4), which generally increases protein stability/levels in tissues, including the heart. In the heart, β-arrestin 1 appears detrimental, whereas β-arrestin 2 beneficial, for structure and function post-myocardial infarction (MI). It is of note that β-arrestin 2 is only minimally expressed, i.e. barely detectable at the protein level, in human myocardium. β-arrestin 1 is by far the most prominent isoform in human myocardium (11). Post-MI β-arrestin 1 knockout mice also display elevated SERCA2a activity and better contractility than post-MI wild type mice. In addition, reduced cardiac SERCA2a SUMOylation is known to underlie its downregulation in heart failure (HF), decreasing cardiac contractility. Thus, the experiments described herein were designed to investigate a potential involvement of cardiac β₁AR-activated β-arrestins in regulation of SERCA2a SUMOylation and activity. Methods and compositions for modulation of β-arrestins may provide superior positive inotropic drugs for therapy of heart failure, particularly of heart failure post-myocardial infarction.

### SUMMARY OF THE INVENTION

Heart failure (HF) is the number one killer disease in the western world and new and innovative treatments are needed. The instant invention answers this need by providing novel methods and compositions for treating heart failure by enhancing cardiac contractility by activating β-arrestin-2 to enhance sarco(endo) plasmic reticulum Ca⁺² ATPase (SERCA-2a) small ubiquitin-like modifier-ylation (SUMOlation). These methods and compositions are particularly useful for treatment of chronic heart failure and/or heart failure that develops/occurs post-myocardial infarction.

In one aspect, the instant invention provides methods for treatment of heart failure, chronic heart failure, and heart failure post-myocardial infarction.

In another aspect, the instant invention provides compositions for treatment of heart failure, chronic heart failure, and heart failure post-myocardial infarction.

In one aspect, the instant invention provides a method for enhancing cardiac contractility comprising activating β-arrestin 2. This enhanced cardiac contractility can be achieved by providing a composition including a modulator of β-arrestin, i.e. a 2 β-1 adrenergic receptor (β-1 AR) ligand that induces β-arrestin 2 binding to a β-1 adrenergic receptor, and administering the composition to a patient/subject. This method can be utilized in any patient, human or animal, that would benefit from enhanced cardiac contractility. A “patient” can also be referred to as a “subject.” Activation of β-arrestin 2 can include enhancing the process of sarco(endo) plasmic reticulum Ca⁺² ATPase (SERCA-2a) small ubiquitin-like modifier-ylation (SUMOlation). Enhancement of SERCA-2a small ubiquitin-like modifier-ylation (SUMOlation) enhances, improves, and/or increases cardiac contractility.

Another aspect of the inventive method includes a preliminary step for administering a composition including a nucleic acid encoding cardiac β-arrestin 2 to the patient prior to administering the composition including a modulator of β-arrestin 2 (to the patient). This is to augment expression of β-arrestin 2 in the heart and may improve efficiency of the modulator (of β-arrestin 2).

Another aspect of the invention embodies pharmaceutical compositions for increasing cardiac contractility in patients. These compositions include therapeutically-effective amounts of a β-1 adrenergic receptor (β-1 AR) ligand that induces β-arrestin 2 binding to a β-1 adrenergic receptor and at least one pharmaceutically-acceptable carrier. These ligands preferentially activate and/or stabilize β-arrestin 2 rather than β-arrestin 1. These compositions are useful for treating any patient having heart failure, particularly chronic heart failure or heart failure brought on by myocardial infarction.

The phrase “pharmaceutically-acceptable carrier” refers to an inactive and non-toxic substance used in association with an active substance, i.e. in this invention the active substances are the modulators of β-arrestin 2 , especially for aiding in the application/delivery of the active substance. Non-limiting examples of pharmaceutically-acceptable carriers are diluents, binders, disintegrants, superdisintegrants, flavorings, fillers, and lubricants. Pharmaceutically-acceptable carriers can have more than one function, a non-limiting e.g. a filler can also be a disintegrant. Additionally, pharmaceutically-acceptable carriers may also be referred to as non-medicinal ingredients (NMIs) or pharmaceutically-acceptable excipients.

The phrase “effective amount” refers to the amount of a composition necessary to achieve the composition's intended function.

The phrase “therapeutically-effective amount” refers to the amount of a composition required to achieve the desired function, i.e. enhanced cardiac contractility and/or attenuation/treatment of the symptoms of heart failure.

Another embodiment of the invention provides a method for attenuating the progression of heart failure in a patient, particularly, but not limited to, heart failure progression post-myocardial infarction, comprising administering a therapeutically-effective amount of a composition including a β-1 adrenergic receptor (β-1 AR) ligand that induces β-arrestin 2 binding to a β-1 adrenergic receptor. Attenuating progression of heart failure includes increasing cardiac contractility.

In another aspect, any of the above-described modulators of β-arrestin 2 and pharmaceutically-acceptable carriers can be used in the manufacture of any of the above-described compositions and pharmaceutical/therapeutic compositions.

Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 is a graph showing maximal cardiac SERCA activity in sham-operated (Sham) and in 4-week post-MI (MI) wild type (WT) and βarr1-knockout (KO) adult (3-month-old) mice. *, p<0.05, vs. WT Sham, **, p<0.05, vs. WT MI, n=5 hearts/group. (5)

FIG. 2 shows representative western blots in SERCA2a immunoprecipitates from mouse heart protein extracts. WT: wild type; KO: knockout; IP: immunoprecipitation; IB: Immunoblotting; IgG: Negative control for the co-IP (general rabbit IgG used in the IP instead of a SERCA2a antibody); S-SERCA2a: SUMOylated SERCA2a; βarr2 input: βarr2-overexpressing HEK293 cell lysate (positive control for βarr2 immunoblotting).

FIG. 3A shows data form GST pull-down experiments for the βarr2-SERCA2a interaction. Lane 1: Purified GST-tagged βarr2 (“bait”); Lane 2: SERCA2a overexpression lysate (“prey”); Lane 3: Bait flow-through; Lane 4: Prey flow-through; Lane 5: Bait-prey Elute; Lane 6: Bait-prey Wash.

FIG. 3B shows data from FRET experiments. FRET images of HEK293 cells co-expressing βarr2-YFP and CFP-SERCA2a taken before and after βarr2-YFP bleaching. CFP and YFP fluorescence are indicated by red and green signals, respectively. The increase in CFP fluorescence detected indicates direct interaction between βarr2-YFP and CFP-SERCA2a.

FIG. 3C also shows data from FRET experiments, specifically FRET efficiency (E_{FRET}) calculated for this interaction. HEK293 cells expressing SERCA2a-CFP alone were used as negative control. **, p<0.01, vs. SERCA2a; n=12 cells (βarr2+SERCA2a), n=9 cells (SERCA2a alone).

FIG. 4A shows western blotting in Adrβarr1-or Adβarr2-transfected H9c2 cell lysates to confirm βarr transgene overexpression. Blots in control AdGFP-transfected cells are also shown for comparison. Only βarr1 appears to be expressed endogenously in H9c2 cells (control AdGFP lanes).

FIG. 4B shows representative western blots in SERCA2a immunoprecipitates from these cell lysates to detect interaction with βarr. IP: immunoprecipitation; IB:

Immunoblotting; IgG: Negative control for the co-IP (general rabbit IgG used in the IP instead of a SERCA2a antibody); ICI: Treatment with 10 μM ICI-118,551 for 30 min; Iso: Treatment with 1 μM lisoproterenol for 10 min (in the presence of 10 μM ICI-118,551); Input: Cell lysate from HEK293 cells overexpressing both βarrs.

FIG. 4C shows Western blotting for SUMOylated SERCA2a (S-SERCA2a) in lysates from Adrβarr1-, Adrβarr2-, or control AdGFP-transfected H9c2 cells treated for 30 min with 10 μM ICI-118,551 alone (ICI) or 1 μM Isoproterenol (Iso) in the presence of 10 μM ICI-118,551.

FIG. 5A shows in vivo cardiac function of 3-month-old C57/B16 mice at 21 days post-sham operation (Sham) or surgical myocardial infarction (MI). AdGFP or Adβarr2 was injected directly into the murine left ventricular free wall at the time of the MI. Ejection fraction (EF %) as measured with two-dimensional guided M-mode echocardiography using a 14-MHz transducer (VeVo 770 Echograph, VisualSonics, Inc.) is shown.*p<0.05 vs. Sham, * p<0.05 vs. MI-AdGFP, n=4 mice/group.

FIG. 5B shows representative western blots from cardiac extracts from the mice (FIG. 5A) at 21 days post-gene delivery confirming robust cardiac Adβarr2 transgene expression. No transgene expression occurred in the liver or lungs of the Adβarr2-injected mice (data not shown). Control was H9c2 cell extract overexpressing FLAG-tagged βarr2.

FIG. 6 shows a schematic representation of the proposed roles for βarr2 vs. βarr1 in cardiac β1AE-dependent pro-contractile signaling.

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described compositions, formulations, and methods and any further application of the principles of the invention as described herein, are contemplated as would normally occur to one skilled in the art to which the invention relates.

Heart failure (HF) is the number one killer disease in the western world and new and innovative treatments are needed. Sarco(endo)plasmic reticulum Ca²⁺-ATPase (SERCA)-2a is a crucial, for contractile function, calcium-handling protein expressed in the mammalian myocardium and its downregulation is one of the molecular hallmarks of chronic heart failure (HF). If the mechanisms for activation of (SERCA)-2a can be elucidated, particularly activation by βarrestin proteins, compositions for new inotropic drugs for the treatment of heart failure may be developed.

By studying individual β-arrestin knockout heart extracts, it was found that β-arrestin 2 , but not β-arrestin 1, interacts with SERCA2a in the mouse heart in vivo, promoting the latter's SUMOylation and activity. This interaction is direct, as indicated by pull-down and FRET experiments. Finally, via in vitro studies in the cardiomyocyte-like cell line H9c2, it was found that this interaction is both β₁AR-, and beta-agonist-specific, and leads to increased Ubc9-dependent SERCA2a SUMOylation, which, in turn, acutely enhances SERCA2a activity in H9c2 cells. These results suggest that β-arrestin 2 , presumed to also decrease cardiac function by desensitizing βARs, may actually (directly) enhance cardiac contractility, thereby opposing β-arrestin 1 in that regard.

### Methods

Cardiac SERCA2a has been shown to undergo SUMOylation by SUMO1, leading to an increase or upregulation of its activity. This process is downregulated in heart failure (1).

In the heart, βarr1 appears detrimental by reducing function and survival, whereas βarr2 is beneficial by decreasing cardiac inflammation and increasing function and survival in post-myocardial infarction heart failure (5, 6).

The rat cardiomyoblast cell line H9c2 was used, which expresses endogenously both β₁-and β₂ARs and SERCA2a (7, 8).

These experiments were designed to elucidate a potential involvement of cardiac β₁AR-activated βarrs in regulation of SERCA2a SUMOylation and activity.

Cardiac SERCA activity measurements: Cardiac SERCA activity was measured as described (5). Briefly, crude ventricular membranes were prepared and total ATPase activity was assayed by monitoring the rate of loss of A340 after addition of the membrane preparation to a thermostatically controlled (37° C.) cuvette in a spectrophotometer. Background ATPase activity was determined in the absence of ATP. Ca²⁺-independent ATPase activity was assayed in the presence of 10 mM EGTA instead of Ca²⁺ and subtracted from the total ATPase activity to derive the Ca²⁺-dependent ATPase (SERCA) activity.

Co-immunoprecipitation (co-IP) and western blotting: Co-IP for SERCA2a-βarr interaction was done with anti-SERCA2a antibody attached to Protein A-Sepharose beads, followed by western blotting with antibody against βarr1/2. To examine the levels of SUMOylated SERCA2a, SUMO1 in the SERCA2a immunoprecipitates was blotted from mouse heart or H9c2 cell extracts. Western blotting for Ubc9 in the SERCA2a immunoprecipitates was also done to detect the presence of this SUMO ligase in the co-IPs.

GST pull-down assay & FRET analysis: GST pull-down was performed with a GST protein interaction pull-down kit (Pierce Biotechnology) and FRET analysis in transfected HEK293 cells was performed as described previously (9).

Myocardial Infarction (MI) & In Vivo gene transfer: MI was inflicted on the mice by ligation of the left anterior descending (LAD) coronary artery (5). Direct adenoviral injection (Adβarr2 or AdGFP) into the left ventricular (LV) cavity (2×10¹¹ total particles diluted in 80 μl phosphate-buffered saline) was done while the chest was open (5).

Statistical analyses: Data are presented as mean ±SEM. One- or two-way ANOVA with Bonferroni test was used for analysis of numeric parameters and differences were considered significant at p<0.05.

### Results

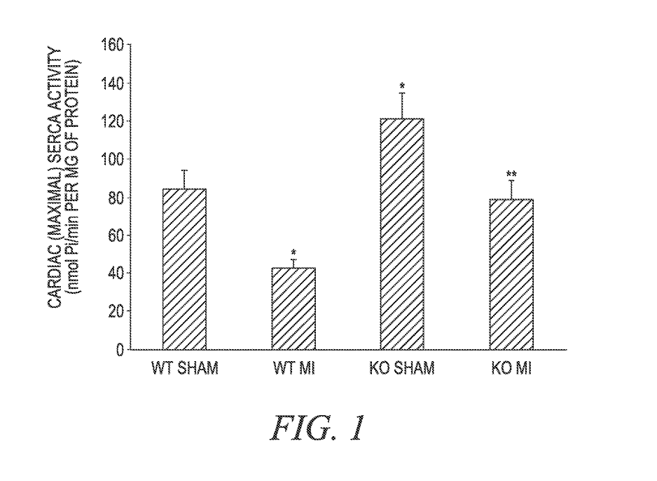

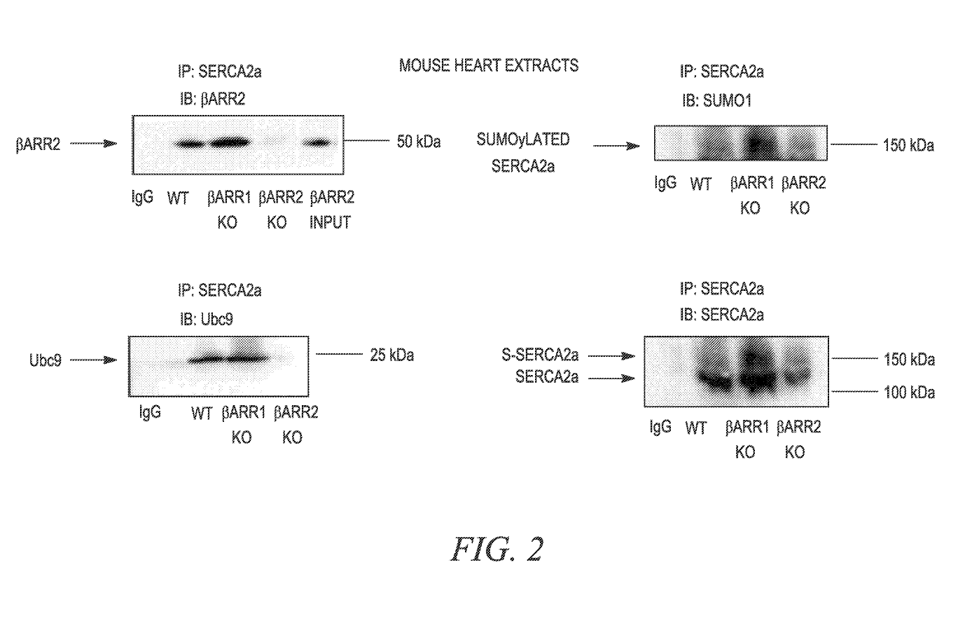

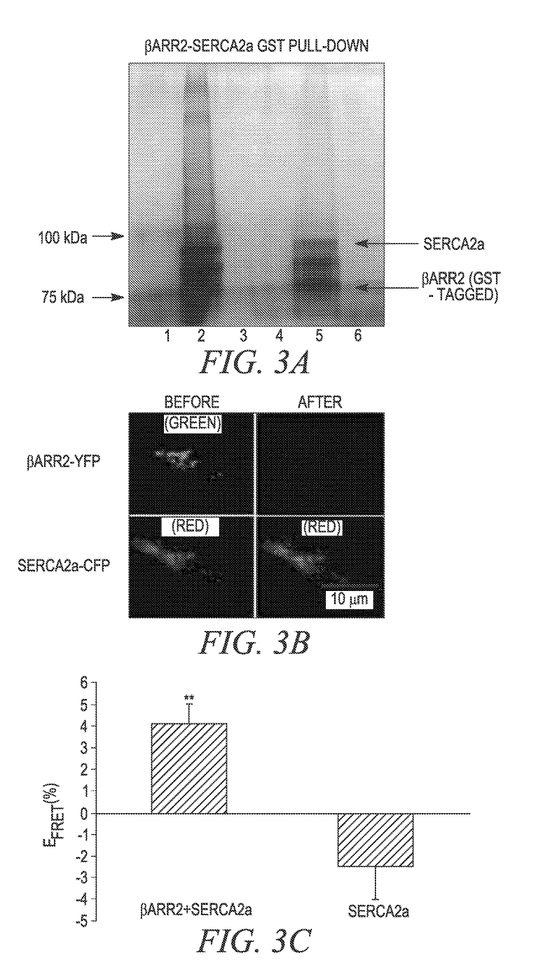

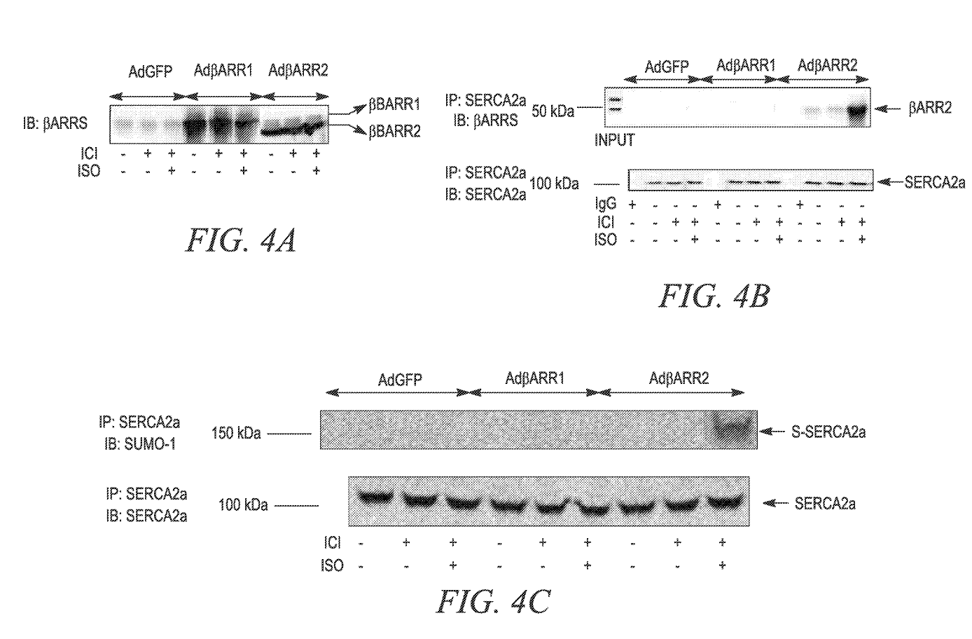

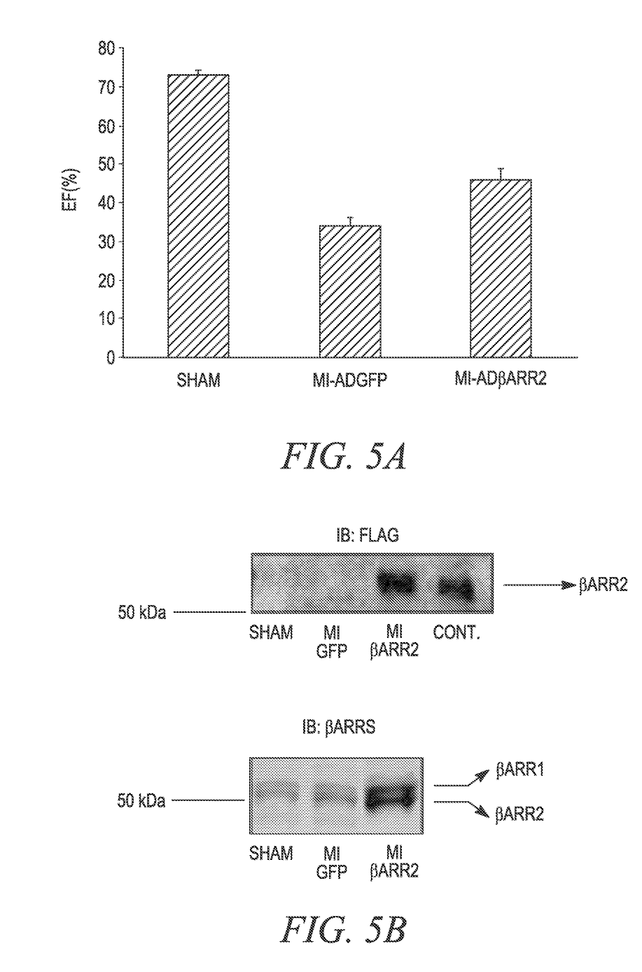

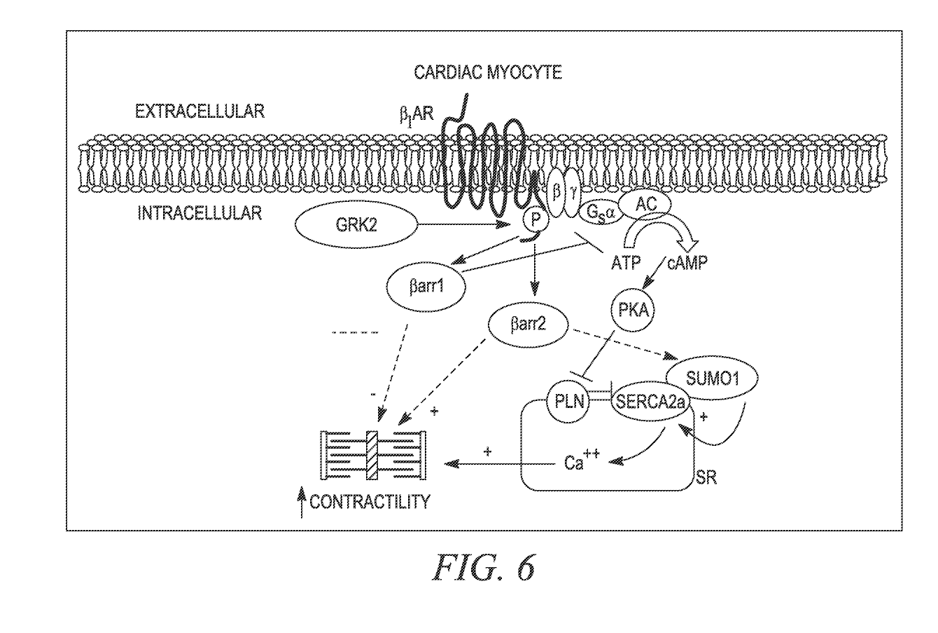

1. βarr1-knockout hearts have increased SERCA activity both normally and post-myocardial infarction (MI). FIG. 1. See also reference (5).
2. βarr2, but not βarr1, interacts with SERCA2a in murine hearts in vivo, enhancing SERCA2a SUMOylation. FIG. 2.
3. The βarr2-SERCA2a interaction is direct. FIGS. 3A-C.
4. βarr2 (but not βarr1) interacts with SERCA2a and enhances its SUMOylation in response to β₁AR activation. FIGS. 4A-C. FIGS. 5A-B. See also reference (10).
5. FIG. 6 shows a schematic representation of the proposed roles for βarr2 vs. βarr1 in cardiac β₁AR-dependent pro-contractile signaling based on the experimental results presented herein.

As evident from the experimental results, cardiac βarr1 diminishes β₁AR-dependent pro-contractile signaling in the heart by terminating its cAMP-mediated signaling (classic receptor desensitization). In contrast, cardiac βarr2, hitherto presumed interchangeable with Paul in that effect, interacts with SERCA2a enhancing SERCA2a SUMOylation and activity, thereby actually promoting β₁AR-dependent pro-contractile signaling in the heart.

Cardiac βarr2 gene transfer in vivo after myocardial infarction (MI) improves cardiac function (and survival), while reducing apoptosis and inflammation in the heart.

These findings suggest that cardiac β₁AR ligands that preferentially induce βarr2 (rather than βarr1 binding to the β₁AR might be superior positive inotropic drugs for therapy of heart failure, particularly chronic heart failure and/or heart failure post- myocardial infarction.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The compositions, therapeutic compositions and methods, pharmaceutical tablets, methods, procedures, and techniques described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

### REFERENCES

1. Kho C, et al. Nature (2011) 477: 601-605

2. Lymperopoulos A, et al. Circ. Res. (2013) 113: 739-753

3. Luttrell L M, Gesty-Palmer D. Pharm. Rev. (2010) 62: 305-330

4. Wyatt D, et al. J. Biol. Chem. (2011) 286: 3884-3883

5. Bathgate-Siryk A, et al. Hypertension (2014) 63: 404-412

6. Watari K, et al. PLOS One (2013) 8: e68351

7. Yano N, et al. Endocrinol. (2008) 149: 6449-6461

8. Ihara Y, et al. BBRC. (2005) 329: 1343-1349

9. Suzuki Y, et al. BBRC. (2013) 430: 1169-1174

10. Zhu W, et al. Mol. Pharmacol. (2013) 84: 227-235

11. Ungerer M, et al. Circ. Res. (1994) 74:206-213

1. A method for enhancing cardiac contractility in a patient, the method comprising:
providing a composition including a modulator of β-arrestin 2;
administering the composition to the patient; and
activating β-arrestin 2 , thereby enhancing cardiac contractility in the patient.

2. The method according to claim 1, wherein the patient has heart failure (HF).

3. The method according to claim 2, wherein the heart failure (HF) is chronic.

4. The method according to claim 2, wherein the heart failure (HF) develops in the patient post-myocardial infarction (MI).

5. The method according to claim 1, wherein the step of activating β-arrestin 2 includes enhancing sarco(endo) plasmic reticulum Ca⁺² ATPase (SERCA-2a) small ubiquitin-like modifier-ylation (SUMOlation).

6. The method according to claim 1, wherein the modulator of β-arrestin 2 is a β-1 adrenergic receptor (β-1 AR) ligand that induces β-arrestin 2 binding to a β-1 adrenergic receptor.

7. The method according to claim 1, further comprising, prior to administering the composition, administering a composition including a nucleic acid encoding cardiac β-arrestin 2 to the patient.

8. A method for enhancing cardiac contractility in a patient, the method comprising:
providing a composition including a modulator of β-arrestin 2;
administering the composition to the patient; and
activating β-arrestin 2 by enhancing sarco(endo) plasmic reticulum Ca⁺² ATPase (SERCA-2a) small ubiquitin-like modifier-ylation (SUMOlation), thereby enhancing cardiac contractility in the patient.

9. The method according to claim 8, wherein the patient has heart failure (HF).

10. The method according to claim 9, wherein the heart failure (HF) is chronic.

11. The method according to claim 9, wherein the heart failure (HF) develops in the patient post-myocardial infarction (MI).

12. The method according to claim 8, wherein the modulator of β-arrestin 2 is a β-1 adrenergic receptor (β-1 AR) ligand that induces β-arrestin 2 binding to a β-1 adrenergic receptor.

13. The method according to claim 8, further comprising, prior to administering the composition, administering a composition including a nucleic acid encoding cardiac β-arrestin 2 to the patient.

14. A pharmaceutical composition for increasing cardiac contractility in a patient, the pharmaceutical composition comprising:
a therapeutically-effective amount of a β-1 adrenergic receptor (β-1 AR) ligand that induces β-arrestin 2 binding to a β-1 adrenergic receptor; and
at least one pharmaceutically-acceptable carrier.

15. A method for attenuating progression of heart failure (HF) by increasing cardiac contractility in a patient having heart failure (HF), the method comprising:
providing the pharmaceutical composition according to claim 14; and
administering the pharmaceutical composition to the patient, thereby increasing cardiac contractility and attenuating progression of heart failure in the patient.

16. The method according to claim 15, wherein the heart failure (HF) is chronic.

17. The method according to claim 15, wherein the heart failure (HF) develops in the patient post-myocardial infarction (MI).

18. The method according to claim 15, further comprising, upon administering the pharmaceutical composition to the patient, activating β-arrestin 2 and enhancing sarco(endo) plasmic reticulum Ca⁺² ATPase (SERCA-2a) small ubiquitin-like modifier-ylation (SUMOlation).

19. The method according to claim 15, further comprising, prior to administering the pharmaceutical composition, administering a composition including a nucleic acid encoding cardiac β-arrestin 2 to the patient.

20-23. (canceled)
